(19) United States
(12) Patent Application Publication
(10) Pub. No.: US 2013/0268037 A1
(43) Pub. Date: Oct. 10, 2013

(54) ELECTRICAL LEAD WITH COUPLING FEATURES

(71) Applicant: NEUROACCESS TECHNOLOGIES, Boston, MA (US)

(72) Inventors: Gregory Schulte, Minneapolis, MN (US); Scott Kokones, Brookline, MA (US); Shivanand Lad, Durham, NC (US); Vincent Owens, Hingham, MA (US); Andrew Firlik, Darien, CT (US)

(73) Assignee: NeuroAccess Technologies, Boston, MA (US)

(21) Appl. No.: 13/832,079

(22) Filed: Mar. 15, 2013

Related U.S. Application Data
(60) Provisional application No. 61/622,337, filed on Apr. 10, 2012.
(60) Provisional application No. 61/712,517, filed on Oct. 11, 2012.
(60) Provisional application No. 61/729,452, filed on Nov. 23, 2012.

(51) Int. Cl. A61N 1/05

(52) U.S. Cl. CPC A61N 1/0551
USPC 607/116

(57) ABSTRACT

An electrical lead including a coupling feature thereon to secure an implantation tool thereto is disclosed.

### CROSS-REFERENCE TO RELATED APPLICATIONS

The present application claims priority to U.S. Provisional Application No. 61/622,337 filed on Apr. 10, 2012 entitled: “Systems and Methods for Stimulating the Spinal Cord;” U.S. Provisional Application No. 61,712,517 filed on Oct. 11, 2012 entitled: “Lead Assemblies, Implantation Tools, Accessory Tools, and Systems and Methods of Using Same;” and U.S. Provisional Application No. 61/729,452 filed on Nov. 23, 2012 entitled: “Positioning Elements and Tools for Adding Positioning Elements to Leads and Catheters,” all of which are incorporated by reference herein in their entirety.

### TECHNICAL FIELD

The present invention relates to systems, devices and methods for positioning an electrical lead in the body.

### BACKGROUND

Paddle-style surgical stimulation leads are commonly used for spinal cord stimulation (SCS) procedures. Common practice involves surgical tools such as forceps to guide and advance the paddle-style leads into the epidural space of the spinal canal. These tools are not ideal as the shape and attachment mechanisms are not specifically designed for the procedure. Therefore, there is a need in the art for an installation tool that can be shaped and manipulated to provide access to the spinal canal. Further, there is a need in the art for an electrical lead that can mate with such an installation tool to secure the lead to the tool during insertion and implantation of the lead.

### SUMMARY

The present invention provides systems, devices and methods for implanting an electrical lead at a target site in a patient's body. In a preferred embodiment, the electrical lead is a surgical paddle-style lead although the electrical lead could be an elongated percutaneous lead. In certain embodiments, the present invention provides systems for implanting an electrical lead comprising an electrical lead positioning tool and an electrical lead. In other embodiments, the present invention provides systems for implanting an electrical lead comprising an electrical lead positioning tool and a simulation lead. In yet other embodiments, the present invention provides electrical leads. In other embodiments, the present invention provides methods of implanting an electrical lead.

In particular, in an embodiment, the present invention provides an electrical lead positioning system comprising an electrical lead positioning tool and an electrical lead. The electrical lead positioning tool comprises a handle and a shaft. The handle comprises a handle body having a proximal end and a distal end. The shaft comprises an elongate body having a proximal portion with a proximal end and a distal portion with a distal end. The proximal end of the shaft extends from the distal end of the handle body. The shaft has a longitudinal x-axis extending between the proximal and distal ends of the shaft body. The distal portion of the shaft body has a substantially non-cylindrical cross-section and a portion of the shaft body comprises a malleable material. The electrical lead comprises an electrical elongate lead body having a proximal end and a distal end, an electrical conductor extending from the proximal end and through the distal end of the lead body, and a substantially flat paddle. The paddle comprises a paddle body extending from the distal end of the lead body. The paddle body has a front side, a back side, a first lateral side, a second lateral side, a proximal end, a distal end, and a length extending between the proximal and distal ends. The paddle further comprises an electrode array comprising an electrode in electrical communication with the electrical conductor. The electrode array is located on a portion of the length of the paddle body.

In another embodiment, the present invention provides a method of positioning an electrical lead on a target site in a patient's body. The method includes providing an electrical lead positioning system including an electrical lead positioning tool and an electrical lead as described above, for example. The method further comprises coupling the electrical lead positioning tool to the electrical lead. The method then includes positioning the paddle on the target site of the patient's body and then removing the tool from the patient's body.

In another embodiment, the present invention provides an electrical lead positioning system comprising an electrical lead positioning tool and a simulation lead. The electrical lead positioning tool comprises a handle and a shaft as describe above, for example. The electrical simulation lead comprises an elongate simulation lead body having a proximal end and a distal end and a substantially flat paddle. The paddle comprises a paddle body at the distal end of the simulation lead body. The paddle body has a front side, a back side, a first lateral side, a second lateral side, a proximal end, a distal end, and a length extending between the proximal and distal ends. In certain embodiments, the system also includes an electrical lead as described above that includes an electrode array comprising an electrode.

In another embodiment, the present invention provides an electrical lead. The electrical lead comprises an elongate electrical lead body having a proximal end and a distal end, an electrical conductor extending from the proximal end and through the distal end, and a substantially flat paddle. The paddle comprises a paddle body at the distal end of the lead body. The paddle body has a front side, a back side, a first lateral side, a second lateral side, a proximal end, a distal end, and a length extending between the proximal and distal ends. The paddle also includes an electrode array comprising an electrode in electrical communication with the electrical conductor. The electrode array is located on a portion of the length of the paddle body. The paddle further includes a pocket located on a length of the paddle body.

In another embodiment, the present invention provides an electrical lead system comprising an elongate electrical lead body having a proximal end and a distal end, an electrical conductor extending from the proximal end and through the distal end, and a substantially flat paddle. The paddle comprises a paddle body at the distal end of the lead body. The paddle body has a front side, a back side, a first lateral side, a second lateral side, a proximal end, a distal end, and a length extending between the proximal and distal ends. The paddle body comprises an electrode array comprising an electrode in electrical communication with the electrical conductor. The electrode array is located on a portion of the length of the paddle body. The electrical lead system also includes a positioning device on the distal end of the paddle body to position the electrode array in a desired location in the patient's body in an operative configuration of the electrical lead.

### BRIEF DESCRIPTION OF THE DRAWINGS

FIG. 1 is a perspective view of an electrical lead positioning system according to an embodiment of the present invention.

FIG. 2 is a perspective view of an implantation tool according to an embodiment of the present invention.

FIG. 3 is a perspective partial view of an electrical lead according to an embodiment of the present invention.

FIG. 4 is a perspective view of an implantation tool inserted into an exemplary coupling feature of a lead according to an embodiment of the present invention.

FIG. 5 is a cutaway partial view of an implantation tool inserted into an exemplary coupling feature of a lead according to an embodiment of the present invention.

FIG. 6 is a partial view of a lead according to an embodiment of the present invention.

FIG. 7A is a perspective view of an implantation tool positioned on a lead according to an embodiment of the present invention.

FIG. 7B is a perspective view of the implantation tool of FIG. 7A inserted into an exemplary receptacle of a lead according to an embodiment of the present invention.

FIG. 8 is a perspective view of a handle of an implantation tool according to an embodiment of the present invention.

FIG. 9 is a perspective partial view of an implantation tool according to an embodiment of the present invention.

FIG. 10 is a perspective partial view of a lead according to an embodiment of the present invention.

FIG. 11A is a top view of a lead according to an embodiment of the present invention.

FIG. 11B is a top view of the lead of FIG. 11A with an implantation tool positioned on the lead in an operative configuration.

FIG. 12 is a perspective view of a lead positioning system with a positioning device disposed on the lead according to an embodiment of the present invention.

FIG. 13 is a perspective partial view of a simulation lead according to an embodiment of the present invention.

### DETAILED DESCRIPTION

The present invention provides systems, devices and methods for positioning a medical device at a target site in a patient's body. In preferred embodiments, the patient is a mammal, such as a human being. In certain embodiments, the medical device delivers a therapy signal to a therapy site in the patient's body. The therapy site can be the same site as the target site or can be adjacent to the target site. The medical device, for example, can be an electrical lead and/or a drug delivery catheter and the therapy signal can be an electrical signal and/or a chemical signal that provides a therapeutic effect to the patient. Although the present invention may be described with respect to SCS where the therapy site is the spinal cord and the target site is the epidural or intradural space of the spinal canal, the present invention can be used for other therapeutic purposes for other parts of a patient's body. For example, the present invention can be used for neuromodulation or other therapies of the brain, including the cortex and specifically the motor cortex; cranial nerves such as the vagus nerve; peripheral nerves such as spinal nerves including the occipital nerve and sacral nerve; and other regions of the nervous system, both the central and peripheral nervous system. The systems, devices, and methods can be used for deep brain leads, gastric leads, vagus nerve leads, peripheral nerve leads including occipital nerve and sacral nerve leads, drug delivery catheters, cardiac catheters, and cardiac stimulation leads.

The disclosure herein may refer to electrical or neural “stimulation” or “modulation.” Such terms include both inhibition and activation of electrical activity in and/or around the therapy site. The disclosure herein also refers to the term “substantially” with respect to certain geometric shapes and configurations. By “substantially” is meant that the shape or configuration of the described component, feature, or element need not have the mathematically exact described shape or configuration, but can have a shape that is recognizable by one skilled in the art as generally or approximately having the described shape or configuration. Also, the disclosure herein refers to an “operative configuration.” This is the configuration of the system when the medical device has been inserted into the patient and is being steered to the target site. Further, as used herein with respect to a described component, the terms “a,” “an,” and “the” include at least one or more of the described component unless otherwise indicated. Further, the term “or” includes “and/or” unless otherwise indicate. In addition, it will be understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

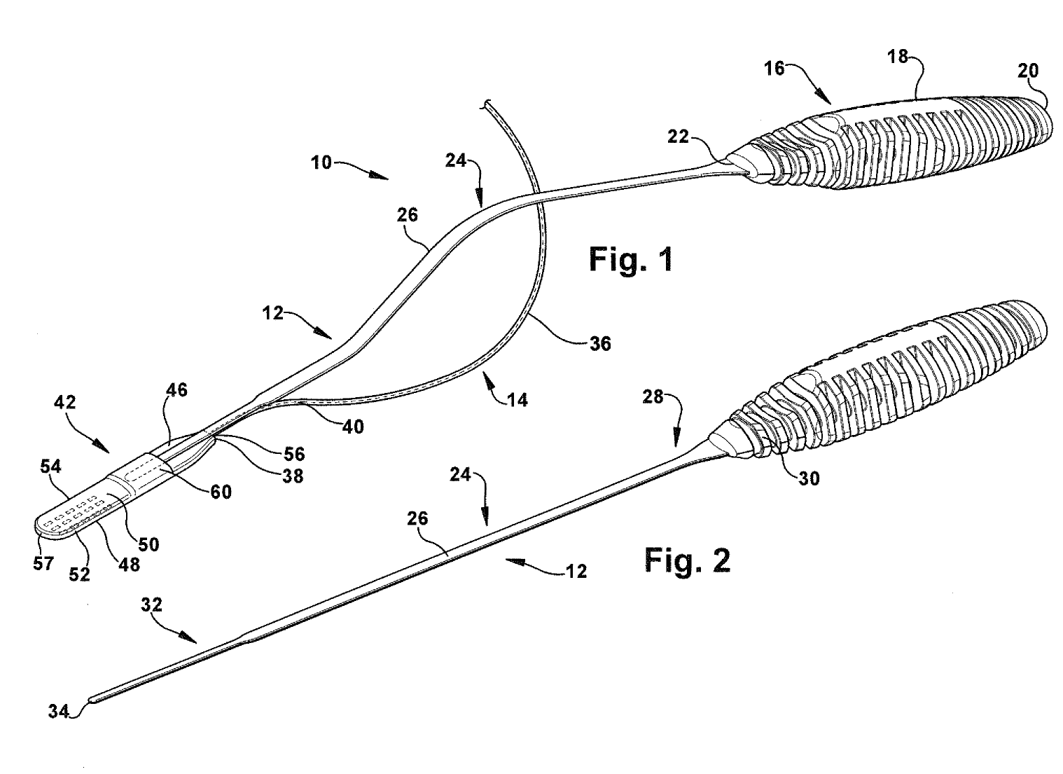

Referring to FIG. 1, in an embodiment the present invention provides an electrical lead positioning system 10 including an electrical lead positioning tool 12 and an electrical lead 14. The present invention also provides an electrical lead 14. Electrical lead positioning tool 12 comprises a handle 16 comprising a handle body 18 having a proximal end 20 and a distal end 22. As also shown in FIG. 2, tool 12 comprises a shaft 24 comprising an elongate body 26 having a proximal portion 28 with a proximal end 30 (shown inside of handle body 18 in FIGS. 1 and 2) and a distal portion 32 with a distal end 34. A longitudinal x-axis extends between proximal and distal ends 30 and 34. Proximal portion 28 extends from distal end 22 of handle body 18. Handle body 18 and shaft 24 can be made of the same or different material. A portion of shaft body 26 comprises a malleable material. In certain embodiments, distal portion 32 is malleable.

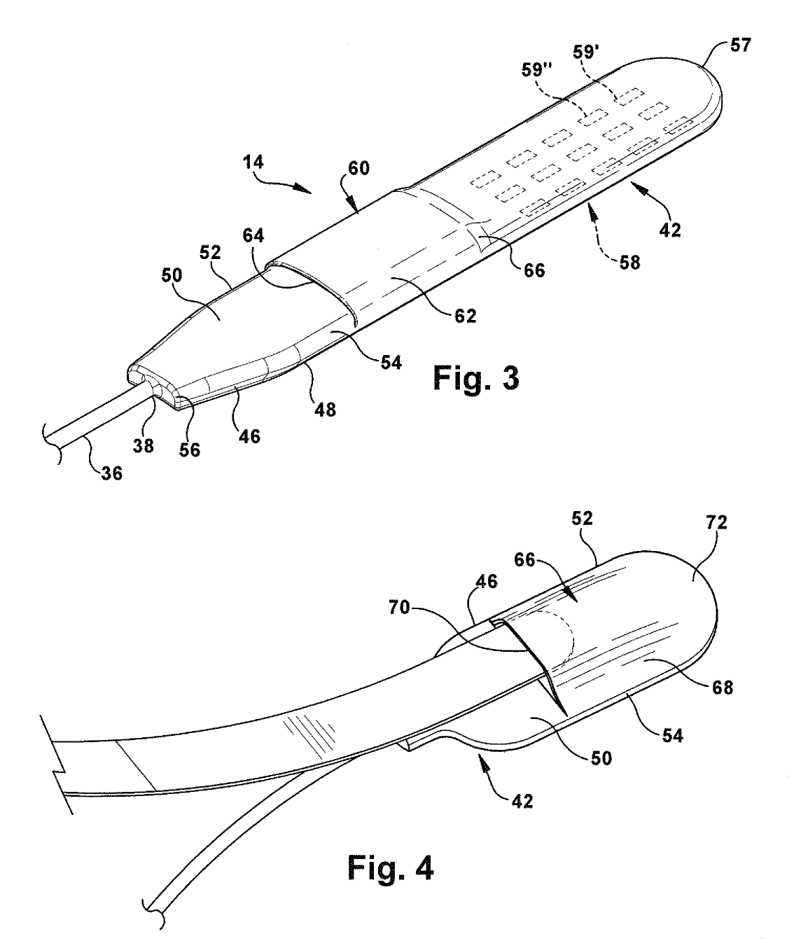

Referring to FIG. 3, in an embodiment, electrical lead 14 comprises an electrical elongate lead body 36 having a proximal end (not shown) and distal end 38. A conductor 40 (shown in FIG. 1) extends from the proximal end and through the distal end 38 of lead body 36. Electrical lead 14 further comprises a substantially flat paddle 42. Paddle 42 comprises paddle body 46 extending from distal end 38 of lead body 36. Paddle body 46 has a front side 48, a back side 50, a first lateral side 52, a second lateral side 54, a proximal end 56, a distal end 57, and a length extending between proximal and distal ends 56 and 57. As described herein, the back side of the paddle body is the non-stimulating face of the paddle body. Paddle 42 also comprises an electrode array 58 comprising an electrode 59 in electrical communication with electrical conductor 40. In preferred embodiments, electrode array comprises a plurality of electrodes 59. Electrode array 58 is located on a portion of the length of front side 48 of paddle body 46. Although in this embodiment, the electrical lead is a surgical paddle-style lead, the electrical lead could also be an elongated percutaneous lead.

In a preferred embodiment, the paddle comprises a coupling feature(s) to securely retain the implantation tool during insertion and implantation of the electrical lead. The coupling feature can be any type of fastener that releasably mates the lead to the distal end of the implantation tool so that the tool is attached to the lead during insertion and implantation and the tool can be removed from the lead after implantation. The fastener can be a male/female fastener, a hook, snap, groove, or any suitable combination thereof. In addition or alternatively, the coupling feature can be a pocket on the paddle body, mechanical key features, magnets, suction, mechanical grasping, or other features to draw the electrical lead taut and to keep the lead from excessive movement. Preferably, the fastener or other coupling feature is located on at least a part of the length of the back side of the paddle body to avoid contact or potential damage to the electrode array, although the fastener or other coupling feature can by on any of the sides of the paddle body. In certain embodiments, the back side of the paddle body comprises a substantially flat and smooth surface and in other embodiments, the back side comprises a roughened and textured surface.

In a preferred embodiment, as shown in FIGS. 1 and 3, the coupling feature is a pocket. In such an embodiment, paddle 42 comprises a pocket 60 on paddle body 46 to securely receive tool 12 or another implantation tool in an operative configuration of tool 12 or another implantation tool. More preferably, pocket 60 is located on at least part of the length of back side 50 of paddle body 46. In particular, in certain embodiments as seen in FIG. 3, pocket 60 is defined by back side 50 of paddle body 46 and cover 62 is integrally molded to back side 50 of paddle body 46. By “integrally molded” is meant that the paddle body and pocket are molded as one piece during manufacturing or the pocket is otherwise not separable from the paddle body using a normal amount of force without damaging the integrity (i.e. tearing) of either the paddle body and/or the pocket. A normal amount of force is the amount of force a user would use to remove a pocket meant to be separated from the paddle without damaging either structure. In such embodiments, the pocket can be fabricated from the same material as the paddle body, such as silicone.

In the embodiment shown in FIGS. 1 and 3, pocket 60 has an open proximal end 64 and a closed distal end 66. In other embodiments, pocket 60 has an open proximal end and an open distal end, thus, essentially forming a slit on paddle 42. In such embodiments, the distal end of an implantation tool preferably comprises a lip that surfaces from the open distal end to ensure the lead is retained in the pocket. In either embodiment, the distal end of the pocket is preferably spaced from the distal end of the paddle body.

Referring to FIG. 4, in other embodiments, paddle 42 comprises a pocket 66 defined by back side 50 of paddle body 46 and cover 68. Cover 68 has an open proximal end 70 and a closed distal end 72. Cover 68 could also have an open distal end. Cover 68 is attached to first lateral side 52 and second lateral side 54 of paddle body 46.

Preferably, a pocket on the paddle body has a substantially non-cylindrical cross-sectional shape. However, the paddle can have other cross-sectional shapes such as rectangular or elliptical. In a preferred embodiment, the pocket is as thin as feasibly possible in order to minimize device bulk and to fit within the target site, such as the epidural space or the intradural space. For example, in certain embodiments, the pocket adds less than about 3 millimeters to the height of the back of the paddle. In other embodiments, the pocket and/or the proximal open end of the pocket has a greater height to assist the paddle in occupying the epidural space and preventing migration. In a preferred embodiment, the pocket is deep enough so that the implantation tool inserted in the pocket has enough room to steer the lead without dislodging from the pocket. In certain embodiments, the pocket has a depth as measured from the proximal end to the distal end of the pocket between about 5 to about 30 millimeters.

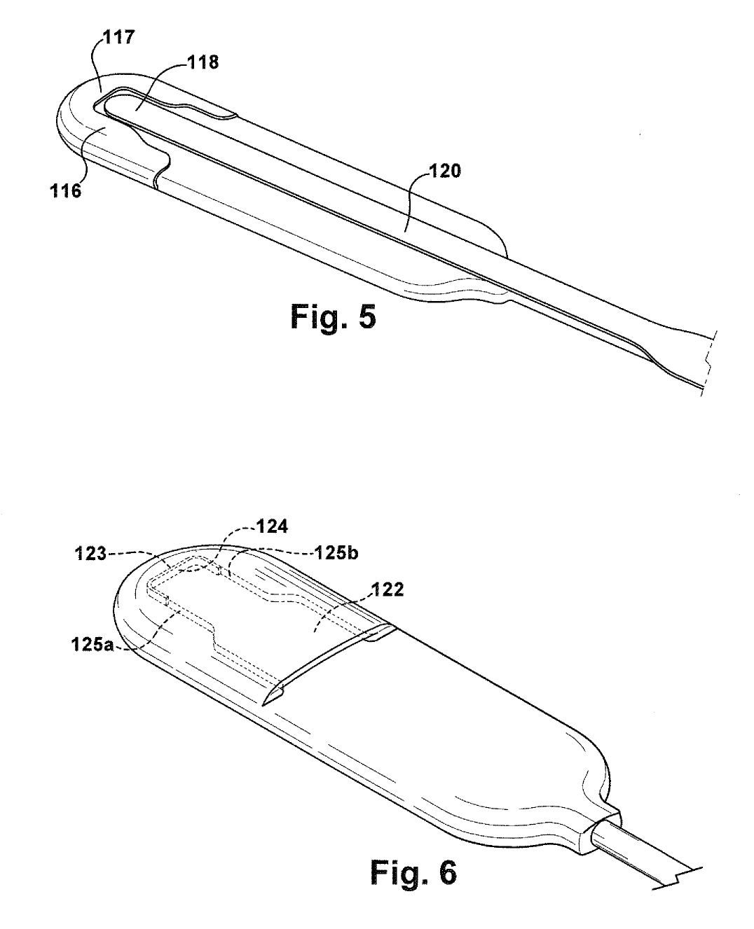

In certain embodiments, where the pocket has a closed distal end, the distal end has a longitudinal thickness that prevents the distal end of an implantation tool that has been positioned in the pocket from poking through the pocket. For example, in certain embodiments, as shown in FIG. 5, the pocket comprises an interior shoulder 116 having a distal wall 117 which the distal end 118 of the implantation tool 120 can abut against in an operative configuration to prevent or reduce the likelihood that the distal end of the tool will pierce the pocket, particularly the distal end of the pocket. Referring to FIG. 6, pocket 122 can comprise a piece 12 at the distal end of pocket 122 such as a metal piece, a hard plastic piece or another piece fabricated from a material different from the material from which the pocket is fabricated. Specifically, in an embodiment, pocket 122 further comprises piece 124 disposed in pocket 122, the piece having a distal wall 123 against which the distal end of the implantation tool abuts against in an operative configuration. In this embodiment, the piece is fabricated from a different material than the pocket. Such a feature could also keep the tool from pushing out through the distal end of the pocket, such as a pocket fabricated from silicone. Such a feature could also be radiopaque so that a user can tell when the tool is fully inserted in the pocket. Further, such a piece can be used in conjunction with an interior shoulder as shown in FIG. 6 to reinforce the shoulder, where the piece fits within the space between and is contiguous with the opposing inner walls 125a and 125b and distal wall 123 of the interior shoulder. Although piece 122 is illustrated in FIG. 6 as having a substantially U-shape, piece 122 could have other shapes so long as the piece prevents or reduces the likelihood that an implantation tool will pierce through the distal end of the pocket. The piece could define apertures so that it could be overmolded into the pocket, such as a silicone pocket.

Preferably, the pocket is not so long that the ability to insert and remove an implantation tool is compromised by forces such as friction. For example, in certain embodiments, where the paddle body has a length of between about 1 to about 2.5 inches, the pocket has a length as measured from the proximal open end to the distal end of between about 0.5 to about 0.7 inches.

Regarding friction between the inside of the pocket and the implantation tool, in certain embodiments to lower possible friction, interior faces of the pocket may include surface treatments such as a plasma coating (including a siloxane based plasma coating) or other treatments that lower friction. The interior faces of the pocket may also be roughened or textured, such as having protrusions, to increase friction and thereby “capture” the implantation tool to hold the implantation tool in place during use. The implantation tool may also include treatments to increase or decrease mating friction to a level that is desirable by the user. For example, in certain embodiments, the shaft body of an implantation tool has a coefficient of friction of about 0.1 or less.

A pocket on a paddle body according to embodiments of the present invention may comprise other features that assist with mating to an implantation tool. For example, the pocket may be tapered from the proximal end to the distal end and/or have a flared open proximal end. The interior faces of the pocket may have a groove, tab, nub, or any suitable combination thereof. Alternatively or in addition, the outside edges of the pocket may also comprise features that assist with implantation tool mating such as a curl or notch, or may be fabricated from materials having different durometers, or any suitable combination of features thereof. Preferably, the open proximal end of the pocket is sufficiently larger than the distal portion of the tool to allow for easy insertion of the implantation tool into the pocket.

In a preferred embodiment, the pocket is fabricated from an elastomer such as silicone so that the pocket may stretch: and conform to the surrounding area of the paddle body. However, the pocket may be fabricated from any suitable biocompatible material, including but not limited to, metals and plastics. Non-limiting examples of materials are stainless steel, MP35N®, Poly Ether Ether Ketone (PEEK), polycarbonate, nylon, polyurethane, silicone, polyurethane/silicone blends, and any suitable combinations thereof.

As mentioned above, a portion of shaft body 26 of implantation tool 12 comprises a malleable material. Preferably, the portion of shaft body 26 includes the distal portion 32 or is the distal portion 32. In other words, the malleable portion of the shaft body can be the entire distal portion or more than the entire distal portion of the tool. The malleability of the implantation tool allows a surgeon or other user to manipulate the distal portion, for example, of the implantation tool to conform to the patient's anatomy. As such, distal portion 32 can be tapered compared to proximal portion 28 to facilitate the user's ability to bend the distal portion of the shaft body. In embodiments of a lead positioning system including an electrical lead with a coupling feature, the malleable portion of the shaft body may be just proximal to the coupling feature so that the coupling feature is not disturbed when the shape of the shaft body is modified. As seen in FIG. 1, in a preferred embodiment, the portion of shaft body 26 that comprises a malleable material is malleable in a plane substantially orthogonal to the longitudinal x-axis. In embodiments where a system is used for SCS, this allows a user to customize the configuration of the implantation tool to navigate to the target site on the same plane as the sagittal plane of the spinal cord. As such, in certain embodiments, the implantation tool has a bend in the shaft body that is substantially orthogonal to the longitudinal x-axis of the shaft body in a pre-operative configuration (when the tool is outside the patient's body and the user is attaching the tool to the electrical lead) or in an operative configuration of the implantation tool. The implantation tool is malleable in the sense that a physician or other user can manipulate the tool to create a bent shape in the tool (as seen in FIG. 1) and then manipulate the tool again to create an un-bent shape (as seen in FIG. 2) without using other instrumentation to accomplish this and without damaging the integrity (i.e. breaking) the tool such that it no longer performs its intended function.

The malleable material can be stainless steel or another biocompatible malleable metallic material. If less than the entire shaft body of the implantation tool is malleable, then the remaining portion of the shaft body is rigid. For example, the remaining portion can be fabricated from PEEK, polycarbonate, ULTEM, or any suitable combination thereof. Alternatively, the entire implantation tool can be fabricated from the same material but sections of the implantation tool can have different properties such that only the shaft body or a portion thereof is malleable. For example, the handle could be fabricated from the same material as the shaft, but could have sufficient thickness such that it is not malleable.

Referring back to FIG. 2, and as described above, a portion of shaft body 26 of implantation tool 12 has a substantially non-cylindrical cross-section in certain embodiments, the distal portion 32 of shaft body 26 has a substantially non-cylindrical cross-section. In a preferred embodiment, the substantially non-cylindrical cross-section is substantially rectangular. In embodiments where the paddle comprises a pocket, preferably an implantation tool's distal cross section is similar in shape to the pocket's cross-section so that the distal end of the implantation tool may be inserted into and mated with the pocket. Further, shaft body 26 is preferably substantially flat and does not have sharp edges in areas where the tool contacts the patient or the electrical lead during use. Also, the distal end of the shaft body can be a fully rounded tip to prevent any damage to a pocket in which the distal end of the shaft body is inserted and to prevent the distal end of the shaft body from “catching” on the pocket edges during insertion of the tool into the pocket. Preferably, the width of shaft body 26 is at least about 1.5 times greater than its height. Preferably, the height is less than about two millimeters. Further, shaft body 26 preferably has a length greater than about three inches.

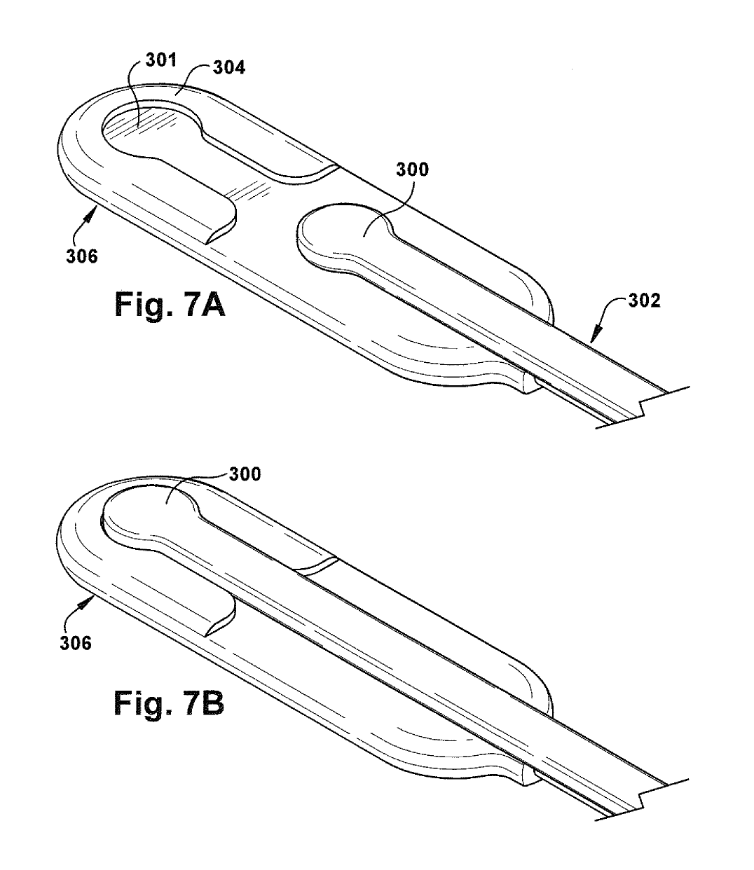

As with the pocket on the paddle, an implantation tool can comprise other features, preferably at the distal end of the implantation tool, that assist with mating to an electrical lead. Such features include, but are not limited to, nubs, notches, a substantially rounded distal end or a distal end without any sharp edges, a taper, and any suitable combination thereof. For example, as seen in FIG. 2 and as mentioned above, distal portion 32 of shaft body 26 can be tapered compared to proximal portion 28. This can be the case with other implantation tools as well used with electrical leads of the present invention. Also, as described above, an implantation tool may be treated or manufactured to increase or decrease mating friction with an electrical lead by way of a surface coating, surface treatment, overmolding, knurling, tumbling, or blasting with media. An implantation tool may also have other features such as nubs, for example, on the shaft body to spread out the pushing force applied by the implantation tool against the lead during use. The nubs can be ridges or bumps, for example, just proximal to the paddle. The nubs can provide some pushing force on the proximal surface of the paddle body to spread out the force so that not all of the pushing force is applied to the distal end of the pocket. In addition, an implantation tool may assume an engaged state and a retracted state. Mechanical features could expand based on activation at the tool's proximal end. This could increase friction or there could be a receptacle at the distal end of the pocket that mates with a complimentary protrusion on the tool to create a temporary mechanical lock that could assist with both insertion and steering. For example, the distal end of the tool could have a spherical enlargement that is a little larger than the shaft width. The pocket then would also have a section at its distal end that tapers and then also opens into a complimentary spherical receptacle. Therefore, when the tool is inserted into the pocket there is some resistance against the tool and then the tool “pops” into the receptacle when fully inserted. This provides both a tactile confirmation of full insertion as well as extra holding force against the implantation tool. An example of such a mechanical engagement between an implantation tool and the pocket is illustrated in FIGS. 7A and 7B. The distal portion 300 of implantation tool 302 has a bulbous shape and the distal portion 304 of pocket 306 has a complimentary bulbous receptacle 301 as seen in FIG. 7A so that when distal portion 300 of implantation tool 302 engages distal portion 304, the two components mate to secure and releasably lock implantation tool 302 to pocket 306 as seen in FIG. 7B. The walls surrounding and defining receptacle 301 can be fabricated from an elastomeric material such as silicone so that the implantation tool securely mates with pocket 306 in an operative configuration. The amount of force it takes to separate the implantation tool from the pocket can be adjusted by varying, for example, the interference fit between the two components. FIG. 7A illustrates an example of a passive engagement between the implantation tool and the pocket but in other embodiments, there could be more of an active engagement. For example, the tool shaft body can have a spring pin at its distal end. There can be a button or other mechanism on the tool's handle that relaxes the spring pin. Therefore, the user can control the activation of the mechanical feature that “locks” the tool to the paddle.

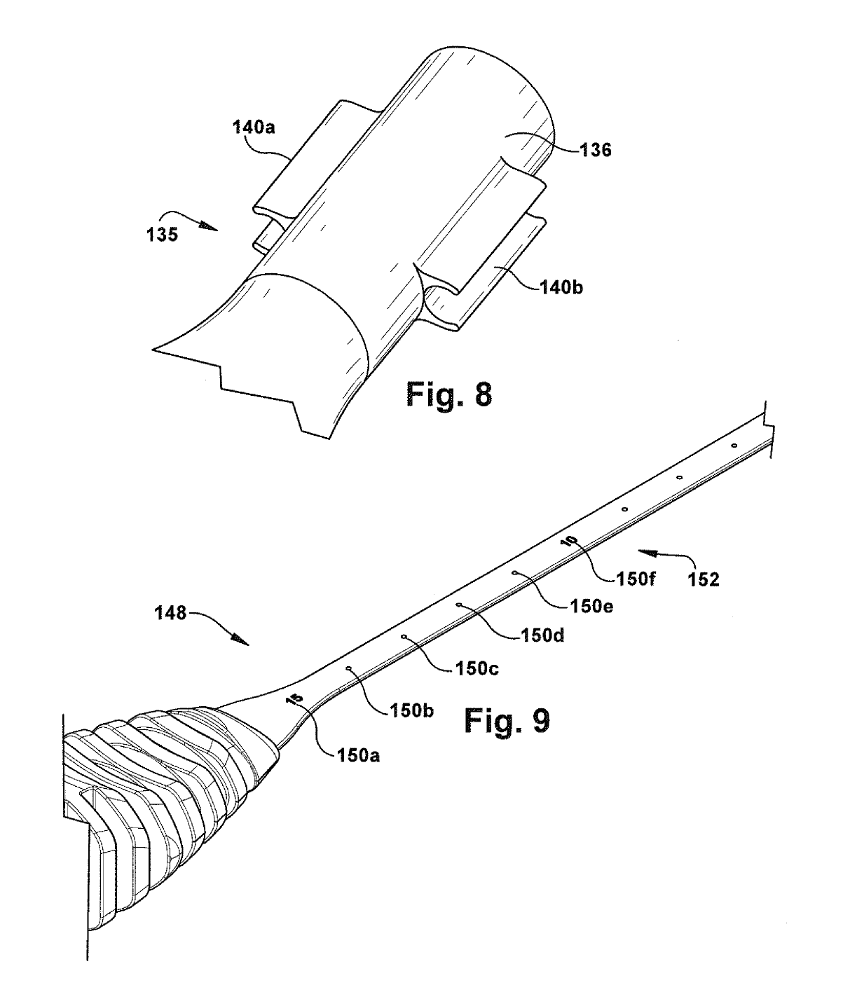

Referring to FIG. 8, in certain embodiments, an implantation tool 135 has a grip 140 on the shaft and/or the handle that releasably holds the lead body or bodies during the implantation procedure to avoid the lead bodies from interfering with the procedure. The grips are depicted in FIG. 8 as being C-shaped clips located on opposing sides of the implantation tool handle 136 but could be located on the shaft body and could be any structure to which the lead body or bodies could be releasably fastened such as clasps, grooves, hooks, or any combination thereof so long as the lead bodies are not damaged.

Referring to FIG. 9, in certain embodiments, an implantation tool 148 comprises measurement markings 150, preferably on the malleable portion of the shaft body 152. Such measurement units or indications allow the user to bend the tool at a spot on the shaft body similar to a spot where the shaft body was bent previously, for example, or to otherwise bend the tool at a spot relative to another spot where the tool was bent. The markings can be separated from one another by any suitable distance and any suitable number of markings can be disposed on shaft body 152. For example, in FIG. 9, the markings include dots (150b-e) separated by one centimeter and include numerical values (150a and f) separated by five centimeters.

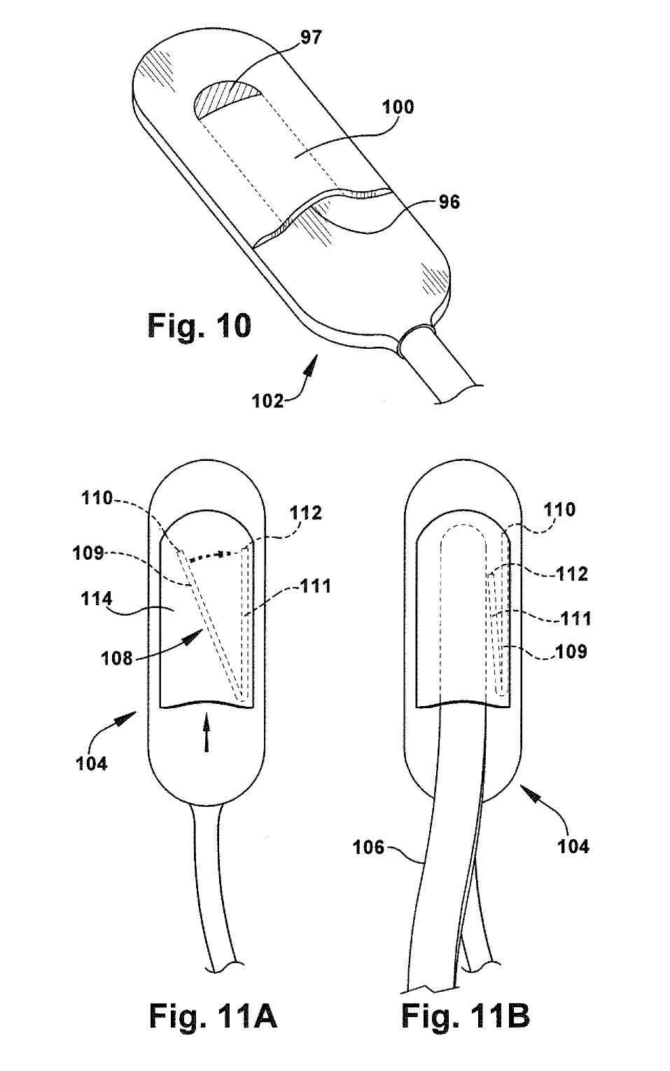

In certain embodiments, the distal end of an implantation tool that engages the back side of the paddle body comprises a metal element or metal-impregnated element such that it is radiopaque and therefore visible under fluoroscopy. In addition or alternatively, the components of an electrical lead comprise a radiopaque marker that allows the system to be visualized under fluoroscopy. For example, referring to FIG. 10, the proximal open end 96 and/or the distal end 97 of pocket 100 of electrical lead 102 can comprise radiopaque markers to assist with alignment of the implantation tool into pocket 100. Non-limiting examples of radiopaque markers include a metal wire, barium loaded silicone, radiopaque silicone inks, or any suitable combination thereof. Referring to FIGS. 11A and 11b, in another embodiment, an electrical lead 104 comprises an elastic metal wire 108 disposed inside of a pocket 114. Wire 108 has a first portion 109 with a first end 110 and a second portion 111 with a second end 112. In a resting position when an implantation tool 106 is not inserted in pocket 114, first portion 109 of wire 108 rests diagonally inside pocket 114 as shown in FIG. 11A. When implantation tool 106 is properly inserted into pocket 114 under fluoroscopy, the user is notified of proper insertion because first portion 109 bends laterally and distally to accommodate tool insertion as shown in FIG. 11B. In another embodiment, the metal wire is a metal loaded plastic flap or other component that is radiopaque and whose movement may be identified by the proper insertion of the tool into the paddle lead mating pocket. For example, a radiopaque identifier can be a barium loaded silicone flap that hangs over the pocket's proximal end. When the tool is inserted, the flap folds into the pocket with the tool, notifying the user under fluoroscopy that the tool is in the pocket.

In certain embodiments the handle of the implantation tool is non-autoclavable so that a physician cannot try to re-use an implantation tool that has been previously employed for another procedure.

In certain embodiments, the present invention provides an electrical lead positioning system with a positioning device mounted to the distal end of the paddle of the electrical lead. Such a device optimally positions electrodes at the target site by urging the electrodes into closer contact with the therapy site. Further details regarding a positioner device are disclosed in co-pending application entitled: “Electrical Lead Positioning Systems and Methods” (Ref. No.: NAT-021916-US-ORD), filed on Mar. 15, 2013 and incorporated by reference herein.

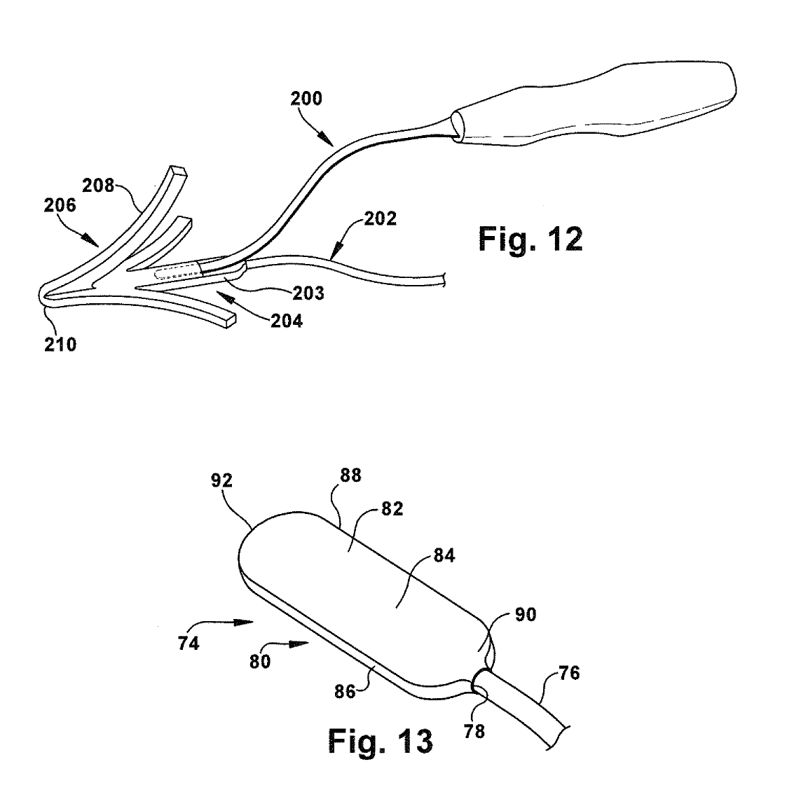

For example, as shown in FIG. 12, electrical lead positioning system can comprise an implantation tool 200 and an electrical lead 202 according to any of the embodiments described above. A positioning device 206 can be mounted on the lead by a user or can be molded or otherwise pre-formed on the distal end of paddle 204. Device 206 has an arm 208 radiating from paddle body 203. In a non-deployed configuration of positioning device 206 (when it is inside of an introducer or otherwise not exposed to the patient's body), the arm 208 is flexible enough to extend radially inward towards paddle 204 and in a deployed configuration (when the electrical lead is ready to be implanted), the arm 208 is stiff enough to extend radially outward away from paddle 204. The arm can be one arm or a plurality of arms and the positioning device can be one positioning device or a plurality of positioning devices. A positioning device can be integrally coupled to paddle 204 such that they are molded together as one-piece during manufacturing or otherwise not separable using a normal amount of force without damaging the integrity (i.e. tearing) of either the paddle or the positioning device. A normal amount of force is the amount of force a user would use to remove a positioning device meant to be separated from the paddle without damaging either structure. In certain embodiments, the positioning device is mounted on to the paddle by a user (e.g. either directly or indirectly using a mounting tool) and can be placed on the distal end or other portion of the electrical lead by a user before or during implantation of the lead.

In certain embodiments, at least one arm extends posteriorly in a deployed configuration. Such a configuration may be advantageous in SCS or other types of therapy applied to the spinal cord where the electrical lead is implanted epidurally or intradurally, as the at least one arm extending posteriorly urges electrodes into closer contact with the spinal cord. In certain embodiments, at least one arm, and preferably two arms, extends laterally in a deployed configuration. Such a configuration may be advantageous as the laterally extending arm helps keep the paddle centered adjacent to the spinal cord. The arms should be configured to not harm surrounding tissue such as the spinal cord or other tissue. As such, in certain embodiments, none of the arms extend anteriorly in a deployed configuration. Such a configuration may be advantageous since none of the arms would potentially drive into the spinal cord. Of course, in embodiments where the arm is a plurality of arms, the positioning device can have any suitable combination of the above configurations. In a preferred embodiment, the positioning device comprises a plurality of arms radiating from the paddle body, the plurality of arms comprising a first laterally extending arm, a second laterally extending arm and no anteriorly extending arm. In certain embodiments, the first laterally extending arm is separated by about 180 degrees from the second laterally extending arm. In embodiments where the plurality of arms includes a posteriorly extending arm, preferably the first laterally extending arm is separated from the posteriorly extending arm by about 90 degrees, the posteriorly extending arm is separated from the second laterally extending arm by about 90 degrees and the second laterally extending arm is separated from the first laterally extending arm by about 180 degrees. In preferred embodiments, the positioned device includes at least three arms and the angle between at least two of the arms is no greater than about 120 degrees. The terms “laterally,” “anteriorly” and “posteriorly” are used herein with respect to the anatomical directions of a human body in a standard anatomical position as is known in the art.

The arm of the positioning device has a length of less than about three centimeters but can be trimmed by the user if it is desired to fixate the lead more laterally, for example. In general, the angle between the first and/or second lateral side of the paddle body and the arm in a deployed configuration should not be so low that the arm is substantially flush with the paddle body thereby providing insignificant outward force against adjacent tissue. In addition, the angle should not be so great that the arm essentially extends cranially and the paddle body does not resist movement upon application of a pulling force to the distal end of the lead. In certain embodiments, the angle between the first or second lateral side of the paddle body and the arm is between about 10 degrees and about 90 degrees in a deployed configuration. In order to be viewed under fluoroscopy, the arm of the positioning device is preferably radiopaque.

In another embodiment, the present invention provides a simulation lead that can be used with an electrical lead positioning system or an electrical lead according to any of the embodiments of the present invention. The paddle and lead body or just the paddle of the simulation lead has the same profile as the electrical lead ultimately implanted in the patient's body but has no electrode array. In particular, in an embodiment, the present invention provides an electrical lead positioning system that includes an electrical lead positioning tool according to any of the embodiments described above and al simulation lead. As seen in FIG. 13, simulation lead 74 comprises an elongate simulation lead body 76 having a proximal end (not shown), a distal end 78, and a substantially flat paddle 80. Paddle 80 comprises a paddle body 82 at a distal end 78 of simulation lead body 76. Paddle body 82 has a front side (not shown), a back side 84, a first lateral side 86, a second lateral side 88, a proximal end 90, a distal end 92 and a length extending between proximal end 90 and distal end 92. A pocket or other coupling feature may be located on the back side 84 of paddle body 82 for receiving and mating with an implantation tool. Simulation lead 74 can serve to clear the pathway in a patient's body for eventual electrical lead placement as described below. In certain embodiments, the simulation lead body and paddle are made from materials that have the same durometer as the electrical lead body and paddle. In embodiments where the simulation lead body has the same diameter as the electrical lead body, the proximal end of the simulation lead body can have a stopper, such as a round plug to assist with retrieval of the simulation lead and/or to present a user from trying to couple the simulation lead to another device such as an extension lead or energy source (such as a pulse generator).

The present invention also provides methods of positioning an electrical lead on a target site in a patient's body. An exemplary method comprises providing an electrical lead positioning system comprising an electrical lead positioning tool and an electrical lead, coupling the tool to the electrical lead, inserting the system into the patient's body, positioning the paddle of the electrical lead on the target site, and removing the tool from the patient's body. In a preferred embodiment, the target site is in the spinal canal and the method is used for spinal cord stimulation. For example, the target site could be in the epidural space or intradural space of the spinal canal. The target site could also be the brain for neurostimulation. Once the target site is reached, a therapy signal (such as an electrical or chemical signal) can be delivered to the therapy site, which can be the same site as the target site or a site adjacent to the target site. In the case of SCS, high frequency stimulation can be delivered via the electrical lead.

One exemplary method of using an electrical lead positioning system to implant an electrical lead in an epidural space of a patient includes the following steps. First a simulation lead may be used to clear the pathway in the body for eventual electrical lead placement. The simulation lead can have a profile similar to the electrical lead. The implantation tool is then inserted into a lead pocket, or attached to another coupling feature, outside of the patient's body. The electrical lead is guided into position. The implantation tool can provide pushing forces on the bottom of the pocket in order to advance the lead into the epidural space. Preferably, the implantation tool is fabricated from metal or has a radiopaque marking so that its position may be identifiable under fluoroscopic imaging. The electrical lead may also have a radiopaque marker in order to determine if the tool is fully inserted into the pocket and to help locate the pocket's proximal open end. Once the tool is withdrawn, the tool can be re-inserted for lead re-positioning or adjustment using tactile sensation and/or fluoroscopic imaging. Coupling features previously described on the tool or electrical lead pocket or both may assist with re-engagement of the tool to the electrical lead.

The foregoing description and examples have been set forth merely to illustrate the invention and are not intended as being limiting. Each of the disclosed aspects and embodiments of the present invention may be considered individually or in combination with other aspects, embodiments, and variations of the invention. Further, while certain features of embodiments of the present invention may be shown in only certain figures, such features can be incorporated into other embodiments shown in other figures while remaining within the scope of the present invention. In addition, unless otherwise specified, none of the steps of the methods of the present invention are confined to any particular order of performance. Modifications of the disclosed embodiments incorporating the spirit and substance of the invention may occur to persons skilled in the art and such modifications are within the scope of the present invention. Furthermore, all references cited herein are incorporated by reference in their entirety.

What is claimed is:

1. An electrical lead comprising:
an elongate electrical lead body having a proximal end and a distal end;
a conductor extending from the proximal end and through the distal end of the lead body;
a substantially flat paddle comprising:
a paddle body at the distal end of the lead body, the paddle body having a front side, a back side, a first lateral side, a second lateral side, a proximal end, a distal end, and a length extending between the proximal and distal ends;
an electrode array comprising an electrode in electrical communication with the electrical conductor, the electrode array located on a portion of the length of the front side of the paddle body; and
a pocket located on a length of the paddle body, the pocket comprising a radiopaque material.

2. The electrical lead of claim 1, wherein the pocket has a radiopaque open proximal end, a radiopaque distal end, or both.

3. The electrical lead of claim 1, wherein the pocket comprises radiopaque markers to assist with alignment of an implantation tool into the pocket.

4. The electrical lead of claim 1, wherein the pocket is located on a portion of the length of the back side of the paddle body.

5. The electrical lead of claim 4, wherein the pocket is defined by the back side of the paddle body and a cover integrally molded to the back side of the paddle body, the cover having an open proximal end and a closed distal end, the closed distal end of the cover spaced from the distal end of the paddle body.

6. The electrical lead of claim 4, wherein the pocket is defined by the back side of the paddle body and a cover integrally molded to the back side of the paddle body, the pocket having an open proximal end and an open distal end, the open distal end of the pocket spaced from the distal end of the paddle body.

7. The electrical lead of claim 4, wherein the pocket is defined by a cover and the back side of the paddle body, the cover having an open proximal end and a closed distal end, the cover attached to the first lateral side and second lateral side of the paddle body.

8. The electrical lead of claim 1, wherein the pocket has a substantially non-cylindrical cross-section.

9. An electrical lead comprising:
an elongate electrical lead body having a proximal end and a distal end, a conductor extending from the proximal end and through the distal end;
a substantially flat paddle comprising:
a paddle body at the distal end of the lead body, the paddle body having a front side, a back side, a first lateral side, a second lateral side, a proximal end, a distal end, and a length extending between the proximal and distal ends;
an electrode array comprising an electrode in electrical communication with the electrical conductor, the electrode array located on a portion of the length of the front side of the paddle body; and
a pocket located on a length of the paddle body, the pocket having an open proximal end, a distal end, and depth between the proximal and distal ends of about 5 to about 30 millimeters.

10. The electrical lead of claim 9, wherein the pocket is located on a portion of the length of the back side of the paddle body.

11. The electrical lead of claim 10, wherein the pocket is defined by the back side of the paddle body and a cover integrally molded to the back side of the paddle body, the cover having an open proximal end and a closed distal end, the closed distal end of the cover spaced from the distal end of the paddle body.

12. The electrical lead of claim 10, wherein the pocket is defined by the back side of the paddle body and a cover integrally molded to the back side of the paddle body, the pocket having an open proximal end and an open distal end, the open distal end of the pocket spaced from the distal end of the paddle body.

13. The electrical lead of claim 10, wherein the pocket is defined by a cover and the back side of the paddle body, the cover having an open proximal end and a closed distal end, the cover attached to the first lateral side and second lateral side of the paddle body.

14. The electrical lead of claim 9, wherein the pocket has a substantially non-cylindrical cross-section.

15. The electrical lead of claim 9, wherein the pocket adds less than 3 millimeters to the height of the back of the paddle.

16. An electrical lead comprising:
an elongate electrical lead body having a proximal end and a distal end, a conductor extending from the proximal end and through the distal end; and
a substantially flat paddle comprising:
a paddle body at the distal end of the lead body, the paddle body having a front side, a back side, a first lateral side, a second lateral side, a proximal end, a distal end, and a length extending between the proximal and distal ends;
an electrode array comprising an electrode in electrical communication with the electrical conductor, the electrode array located on a portion of the length of the front side of the paddle body; and
a pocket located on a length of the paddle body, the pocket having a flared open proximal end.

17. The electrical lead of claim 16, wherein the pocket is located on a portion of the length of the back side of the paddle body.

18. The electrical lead of claim 17, wherein the pocket is defined by the back side of the paddle body and a cover integrally molded to the back side of the paddle body, the cover having an open proximal end and a closed distal end, the closed distal end of the cover spaced from the distal end of the paddle body.

19. The electrical lead of claim 17, wherein the pocket is defined by the back side of the paddle body and a cover integrally molded to the back side of the paddle body, the pocket having an open proximal end and an open distal end, the open distal end of the pocket spaced from the distal end of the paddle body.

20. The electrical lead of claim 17, wherein the pocket is defined by a cover and the back side of the paddle body, the cover having an open proximal end and a closed distal end, the cover attached to the first lateral side and second lateral side of the paddle body.

21. The electrical lead of claim 16, wherein the pocket has a substantially non-cylindrical cross-section.

22. The electrical lead of claim 16, wherein the pocket has a closed distal end, the distal end having a longitudinal thickness that prevents the distal end of an implantation tool securely positioned in the pocket in an operative configuration from exiting the pocket.

23. The electrical lead of claim 16, further comprising an interior shoulder in the pocket, the interior shoulder having a distal wall which a distal end of an implantation tool abuts against in an operative configuration.

24. The electrical lead of claim 16, further comprising a piece disposed in the pocket, the piece having a distal wall against which a distal end of an implantation tool abuts against in an operative configuration, the piece fabricated from a different material than the pocket.

25. The electrical lead of claim 16, wherein the distal portion of an implantation tool has a configuration complimentary to the configuration of a distal portion of the pocket so that the distal portion of the implantation tool is releasably locked to the distal portion of the pocket in an operative configuration.

26. The electrical lead of claim 16, wherein the pocket has a distal end and the pocket is tapered from the proximal end to the distal end.

27. A simulation lead comprising:
an elongate simulation lead body having a proximal end and a distal end;
a substantially flat paddle comprising a paddle body at the distal end of the simulation lead body, the paddle body having a front side, a back side, a first lateral side, a second lateral side, a proximal end, a distal end, and a length extending between the proximal and distal ends; and
a pocket located on a length of the paddle body.

28. A system comprising the simulation lead of claim 27 and further comprising an electrical lead comprising:
an elongate electrical lead body having a proximal end and a distal end, a conductor extending from the proximal end and through the distal end;
a substantially flat paddle comprising:
a paddle body at the distal end of the electrical lead body, the paddle body having a front side, a back side, a first lateral side, a second lateral side, a proximal end, a distal end, and a length extending between the proximal and distal ends; and
an electrode array comprising an electrode in electrical communication with the electrical conductor, the electrode array located on a portion of the length of the front side of the paddle body.

29. The system of claim 28, wherein the electrical lead further comprises a pocket located on the paddle body.

30. The system of claim 29, wherein the simulation lead body and paddle of the simulation lead have the same durometer as the electrical lead body and paddle of the electrical lead.

31. The system of claim 29, wherein the back side of the paddle body of the simulation lead has the same shape as the back side of the paddle body of the electrical lead.
